(19) United States
(12) Patent Application Publication
(10) Pub. No.: US 2024/0277892 A1
(43) Pub. Date: Aug. 22, 2024

(54) FABRICATION AND APPLICATION OF FIBER-BASED HYDROGEL USING ALL-AQUEOUS FORMULATIONS

(71) Applicants: VERSITECH LIMITED, Hong Kong (HK); THE TRUSTEES OF PRINCETON UNIVERSITY, Princeton, NJ (US); THE UNIVERSITY OF HONG KONG, Hong Kong (CN)

(72) Inventors: Yuan Liu, Hong Kong (HK); Yanting Shen, Hong Kong (HK); Ho Cheung Shum, Hong Kong (HK); Janine K. Nunes, Princeton, NJ (US); Howard A. Stone, Princeton, NJ (US)

(21) Appl. No.: 18/571,747

(22) PCT Filed: Aug. 3, 2022
(86) PCT No.: PCT/CN2022/109906

§ 371 Date: Dec. 19, 2023

Related U.S. Application Data
(60) Provisional application No. 63/228,686, filed on Aug. 3, 2021.

(51) Int. Cl. A61L 26/00

(52) U.S. Cl. CPC A61L 26/008; A61L 26/0085; A61L 26/0095

(57) ABSTRACT

Provided are a vehicle body assembling method and a vehicle body assembling apparatus which allow a simple configuration in the vicinity of the connecting portion between an upper jig and a lower jig and allow an increase in the efficiency of assembling work (welding work

### TECHNICAL FIELD

Disclosed are fiber-based hydrogels, methods of making fiber-based hydrogels, and methods of using fiber-based hydrogels.

### BACKGROUND

Hydrogels, which are macromolecular networks of polymer chains filled with water, are used in biomedical applications including drug delivery, tissue engineering and regeneration. Fiber-based hydrogels can be formed by flow-induced gelation. When sheared by a flow, microfibers can become topologically entangled, which serve as permanent crosslinks in a connected network filled with water and form a hydrogel. Since the mechanical interlocking mechanism is independent of chemical reactions or interactions, fiber-based hydrogels can be produced and locally applied simply by injection through a syringe needle.

Traditional production processes of microfiber-based hydrogels include the use of oil/aqueous systems, the continuous phase consisting of a mineral oil, which can potentially impair bio-related applications. The organic solvents may induce one or more harmful effects to human cells, protein denaturation, or loss of cell viability. The residual toxic substances can be washed with detergent several times. However, such washing brings more additional processes and/or greatly increases costs, such as chemical material costs, labor costs, disposal costs, etc. and detergents may harm cells.

### SUMMARY

The following presents a simplified summary of the invention in order to provide a basic understanding of some aspects of the invention. This summary is not an extensive overview of the invention. It is intended to neither identify key or critical elements of the invention nor delineate the scope of the invention. Rather, the sole purpose of this summary is to present some concepts of the invention in a simplified form as a prelude to the more detailed description that is presented hereinafter.

To reduce toxic residuals, improve medical safety, and/or decrease the cost of post-processing cleanse, described herein are all-aqueous formulations (and methods of making and/or using thereof) as production materials to fabricate fiber-based hydrogels.

Disclosed herein is a fiber-based hydrogel, an aqueous hydrogel comprising biocompatible microfibers having a high aspect ratio, meaning the ratio of the length to diameter, L/D, in water, the biocompatible microfibers mechanically interlocked. That is, an aqueous hydrogel comprises biocompatible microfibers having a high aspect ratio length to diameter, where the biocompatible microfibers are mechanically interlocked to form a network filled with water.

Also disclosed is the method of making a fiber-based hydrogel (disclosed is the method of making the fibers that will compose a fiber-based hydrogel) involving contacting an aqueous polymer phase containing a photoinitiator with an aqueous solution phase under ultraviolet light exposure and suitable shear forces to generate a fiber-based hydrogel comprising biocompatible microfibers having a high aspect ratio, meaning the ratio of the length to diameter, the biocompatible microfibers mechanically interlocked. The method of making a fiber-based hydrogel is carried out in an all aqueous environment. The photoinitiator is therefore compatible with aqueous systems.

Also disclosed is the method of making fiber-based hydrogels involving changing the fiber aspect ratios to have matching physicochemical properties and controllable drug release rates for precise control over biophysical and biomedical cues to direct endogenous cells. By simply tuning the fiber aspect ratio L/D, the fiber-based hydrogel can tune a considerable range of physicochemical properties and drug release rates.

Also disclosed is the method of making a fiber-based hydrogel with tunable mechanical properties and/or drug release profile, comprising: contacting an aqueous polymer phase comprising a photoinitiator with an aqueous solution phase under ultraviolet light exposure and suitable shear forces to generate biocompatible microfibers having a high aspect ratio length to diameter of at least 100; and, mechanically interlocking from 1% to 7% by dry weight of the biocompatible microfibers in from 93% to 99% by weight water to provide the fiber-based hydrogel.

Also disclosed is the method of using fiber-based hydrogels involving promoting the tissue regeneration through a mice excision skin model. The fiber-based hydrogels promote wound healing with a faster rate of new tissue regeneration and the appearance of de novo regenerated healthy tissue when compared with a commercial gel.

Further disclosed is the use of the fiber-based hydrogel in the preparation of a medicament with controlled drug release rate and/or drug release profile.

Further disclosed is the use of the fiber-based hydrogel of any one of claims 1-9 in the preparation of a medicament with improved wound healing effect and/or tissue generation effect.

To the accomplishment of the foregoing and related ends, the invention comprises the features hereinafter fully described and particularly pointed out in the claims. The following description and the annexed drawings set forth in detail certain illustrative aspects and implementations of the invention. These are indicative, however, of but a few of the various ways in which the principles of the invention may be employed. Other objects, advantages and novel features of the invention will become apparent from the following detailed description of the invention when considered in conjunction with the drawings.

### BRIEF SUMMARY OF THE DRAWINGS

FIG. 1 depicts the microfiber fabrication: (a) bright-field image of all-aqueous jet in the flow-focusing device, (b) synthesized all-aqueous microfibers with very high-aspect-ratio, L/D ˜600, and (c) bright-field image of microfiber.

FIG. 2 depicts suspensions of fibers and hydrogel production: (a) suspension of microfibers, and (b-c) fabrication of reaction-free and interaction-free hydrogel by injecting the suspension from a syringe without and with a standard needle.

FIG. 3 depicts SEM images show porosity and microstructure of dried injectable hydrogel (scale bars, 200 μm in the low-magnification image and 50 μm in the higher-magnification image).

FIG. 4 depicts cell cytotoxicity testing by MTT assay method for (a) fiber suspensions and (b) hydrogel. Both have great biocompatibility.

FIG. 5 depicts cell cytotoxicity testing by estimating cell numbers for fiber suspensions and hydrogel. Both have great biocompatibility.

FIG. 6 depicts the characterizations of the fiber-based hydrogel. Rheological properties G′ and G″ of the fiber-based hydrogel for different fiber aspect ratios showing viscoelasticity as a function of angular frequency, w. Both G′ and G″ increase with L/D.

FIG. 7 depicts the drug release profile of fiber-based hydrogels for different fiber aspect ratios. Fiber-based hydrogels with a larger fiber aspect ratio provide a prolonged drug release time.

FIG. 8 depicts in vivo proof-of-concept of the efficacy and safety of the fiber-based hydrogel: (a) photographs of mice skin wound tissues for the control group, the commercial gel group, and the fiber-based gel with/without drug group on days 0, 4, 8 and 12. The inner diameter of the rubber ring is 1 cm, (b) measured wound healing rate of different four groups for 4, 8, and 12 days.

FIG. 9 depicts PNIPAM fiber suspensions and fiber-based hydrogels production: (a) suspension of the PNIPAM microfibers, and (b) fabrication of the PNIPAM fiber-based hydrogel from a syringe with a standard needle.

### DETAILED DESCRIPTION

Disclosed herein is a new type of fiber-based hydrogel, which is made in oil-free processing using all-aqueous materials. Hydrogels are frequently used in biomedical applications. The methods herein use an aqueous two-phase system to fabricate the fiber-based hydrogel. Advantages of the aqueous two-phase system include one or more non-toxic, safe, and biocompatible to the human body, and possessing many similarities with biological systems. Compared with traditional methods by others, which use toxic organic solvents as raw materials, the methods herein can significantly impact one or more of the following: reduce toxic residuals, improve medical safety, and decrease the cost of post-processing cleanse. Therefore, a hydrogel material made by the new methods herein has a promising impact on biomedical applications and provides a novel carrier platform for the healthcare technology.

Hydrogels, which are macromolecular networks of polymer chains filled with water, are frequently used in biomedical applications including drug delivery, tissue engineering and regeneration. An oil-free, reaction-free, and interaction-free fabrication of a hydrogel for wound-healing applications is described herein. The flow-induced gelation is exhibited by applying stress on a concentrated suspension of microfibers that are highly flexible and possess a very high aspect ratio of at least 100 (such as length/diameter, LID ˜340). In another embodiment, the high aspect ratio of the microfibers is at least 400. In yet another embodiment, the high aspect ratio of the microfibers is at least 2000. In still yet another embodiment, the high aspect ratio of the microfibers is at least 600. When sheared by a flow, these microfibers can become topologically entangled, which we refer to as “mechanical interlocking”, and form a hydrogel. It is noted that chemical cross-linking may or may not occur within an individual microfiber, but chemical cross-linking does not occur between different microfibers.

Since no chemical crosslinking or physical interactions are required, the hydrogels can be conveniently produced and locally applied simply by injection through a syringe needle. However, in the oil/aqueous systems of the traditional production process, the continuous phase consists of mineral oil, which could potentially impair bio-related applications. The organic solvents may induce harmful effects to human cells, protein denaturation or loss of cell viability. Compared with traditional methods by others, which use toxic organic solvents as raw materials, the materials and methods described herein can significantly reduce toxic residuals, improve medical safety, and/or decrease the cost of post-processing cleanse.

The microfibers include one or more of polyurethane acrylate (PUA), gelatin-hydroxyphenylpropionic acid (Gtn-HPA), 4-hydroxybutyl acrylate (4-HBA), poly(ethylene glycol) diacrylate (PEG-DA), poly(ethylene glycol) dimethacrylate (PEG-DMA), alginate, collagen, fibrin, agarose, polylysine (PLL), poly(lactic-co-glycolic acid) (PLGA), amphiphilic triblock poly(p-dioxanone-co-caprolactone)-block-poly(ethylene oxide)-block-poly(p-dioxanone-co-caprolactone) (PPDO-co-PCL-b-PEG-b-PPDO-co-PCL), polybenzimidazole (PBI), regenerated silk fibroin (RSF), poly-(N-isopropyl acrylamide) (poly(NIPAAM)), poly(sulfone) (PSF), polyacrylonitrile) (PSF), polystyrene (PS), fibrin, polyurethane (PU), poly(methylmethacrylate) (PMMA).

Generally, the microfibers are hydrophilic. The microfibers are typically biocompatible and suitable for tissue engineering applications. For example, fiber hydrogels of the embodiments comprise adhesive properties that provide for in vivo applications. That is, a concentrated suspension of the microfibers undergoes irreversible gelation using a simple mechanical process that does not use chemical reactions. The fiber hydrogel exhibits typical properties of a gel: the mechanical properties are consistent with that of a soft viscoelastic solid and it swells in water. The hydrogel forms in situ, so it can be used as an injectable hydrogel, where it forms immediately upon extrusion from a needle (or another extrusion device such as a slit, a pore, an array of pores, an array of needles, etc.) and the disclosed injectable hydrogels from microfiber suspensions can be employed, for the in situ generation of substrates for cells in tissue engineering, as a drug delivery material, and in wound dressings. Other biomedical applications include a surgical sealant and high strength adhesive; a support for nerve regeneration; and a cartilage replacement, among many others.

Herein, using the aqueous two-phase system (ATPS) instead of traditional oil/aqueous systems to fabricate the all-aqueous jets, which is used as a template to synthesize all-aqueous, flexible and biocompatible microfibers. This provides a good basis for the oil-free injectable hydrogel. The unique advantages of aqueous two-phase system include one or more non-toxic, safe, and biocompatible to the human body, and having a wide range of applications in the food industry, chemical systems and biomedical engineering. Therefore, the hydrogel approach described herein based on aqueous two-phase systems opens up a new route towards one or more of improved wound healing, controlling scar tissue formation, and the appearance of a healed wound.

All-aqueous systems have ultralow interfacial tensions, typically ranging from 10⁻⁶ to 10⁻⁴ N/m, and share many similarities with biological systems making them particularly advantageous for bio-related applications. Due to the ultralow interfacial tension of aqueous-aqueous interfaces, the low interfacial tension within ATPS largely favors the formation of all-aqueous jets due to the slow growth of the Rayleigh-Plateau instability, making it form liquid jets with a very high-aspect ratio without any surfactant, which is commonly used for traditional oil/water system. These all-aqueous jets can therefore be used as excellent templates for fabricating water-based microfibers in all-aqueous solutions.

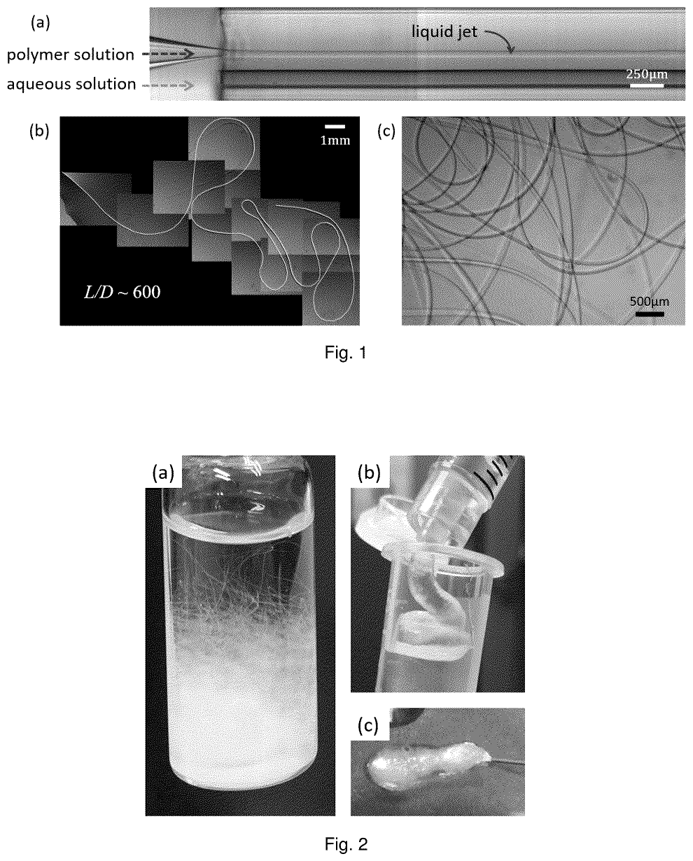

Microfibers are generated by aqueous two-phase system (ATPS) in a flow-focusing microfluidic channel, as shown in FIG. 1(a). All-aqueous jets with a very high-aspect-ratio (typically the ratio of fiber length to fiber diameter, L/D>>100) are achieved by adjusting the flow rates of both the inner and outer phases. After UV exposure, the microfibers are successfully synthesized, as shown in FIG. 1(b-c). The microfiber suspensions are washed by deionized water after collection.

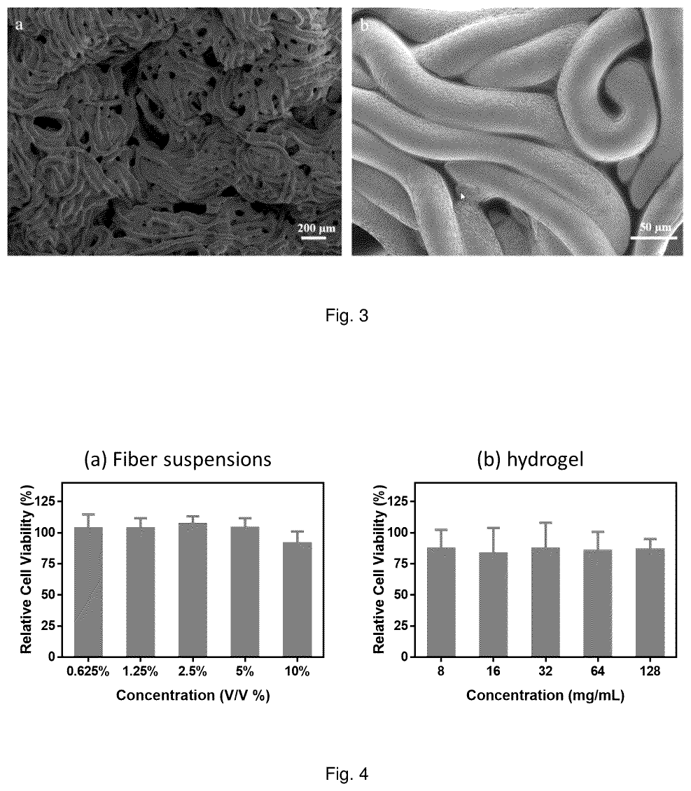

The injectable hydrogel is fabricated by extruding the microfibers suspension from a syringe, as shown in FIG. 2. During the injection process, the fibers deform and create topological entanglements. The structure of the hydrogel is observed through the SEM images of different magnifications in FIG. 3, which shows a dense network of microfibers with a random porous structure. Our hydrogel manufacturing process significantly reduces toxic residuals and the cost of post-processing cleanse.

To demonstrate the advantages of our oil-free, reaction-free injectable hydrogels in biomedical applications and highlight their unique biocompatible properties when compared to chemically and physically crosslinked hydrogels, the cytotoxicity of the crude materials, microfibers and hydrogel, has been tested by two methods, MTT assay and estimating live cell numbers.

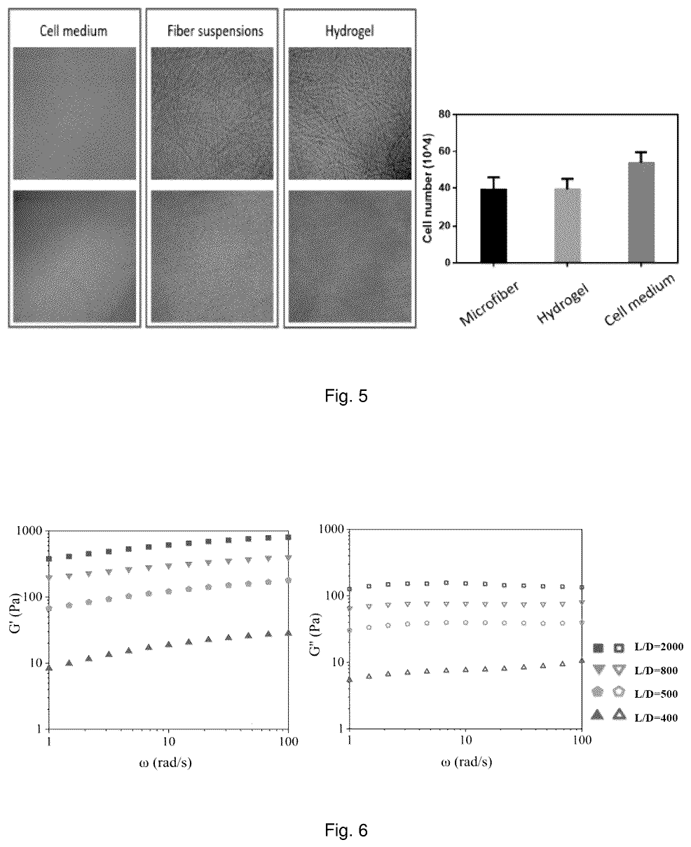

Fiber suspensions and injectable hydrogel are separately co-cultured with cells (L929 mouse fibroblasts) for 24 h. In the MTT assay method, MTT was added into cell to test the quantity of Succinate dehydrogenase (SDH) (OD=570) in Mitochondria from the live cell. Relative cell viability (%)=(ODₘₐₜₑᵣᵢₐₗ−OD_{DI water})/(OD_{cell medium}−OD_{DI water})×100%. The data shows both the microfibers and final hydrogel have great biocompatibility, as shown in FIG. 4. Cell cytotoxicity testing also is checked by estimating cell numbers. In FIG. 5, on the top row, images show cells cultured in the materials at the beginning. After 24 h, cells from each group are cultured with trypsin, and then the numbers of the live cells are calculated from the microscopic images in the bottom row. The results agree with the data by the MTT assay method. Production has great cytocompatibility, which in turn has a promising impact on biomedical applications.

For medical use, such as wound healing, it is essential to match the mechanical properties of hydrogel to that of human body. To verify the mechanical properties of our fiber-based hydrogel, rheological properties of the hydrogel for different fiber aspect ratios (L/D) are measured using a stress-controlled rheometer, as shown in FIG. 6, showing viscoelasticity as a function of angular frequency, ω. Both elastic modulus G′ and viscous modulus G″ increase with fiber aspect ratio. These easily tunable mechanical properties allow the production of a fiber-based hydrogel with a wide range of properties to be constructed for clinical use.

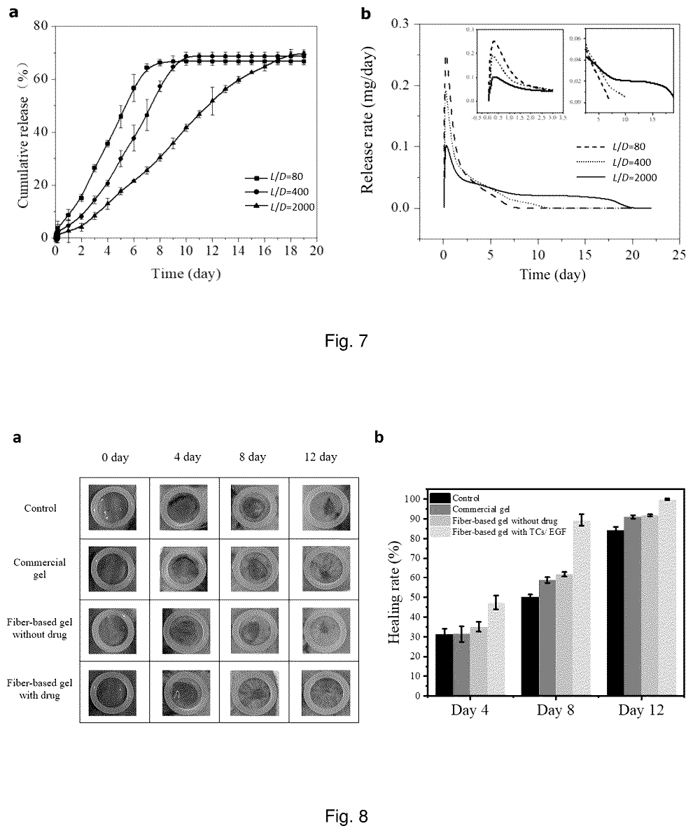

To explore the drug release profile of the fiber-based hydrogel, the released amount of drug from the same volume of fiber-based hydrogels for different fiber aspect ratios was detected in phosphate buffered saline (PBS) by UV spectroscopy, as shown in FIG. 7a, showing that the hydrogel with larger fiber aspect ratio obtains a more sustained drug release profile. The drug release rates of the fiber-based hydrogel with different fiber aspect ratios show that there is a rapid release rate in the early stage as shown in FIG. 7b (inset left figure), which decreases with increasing fiber aspect ratio, followed by a sustained release rate (inset right figure), which increases a small fraction with increasing fiber aspect ratio. For tissue engineering applications, different drugs (e.g., integrins, growth factors, and small molecule medicines) should be presented over different timescales during therapy so that coordinated cellular response can be accurately triggered.

The tissue regeneration efficiency of the fiber-based hydrogel is investigated in vivo by a mice excision skin model. In the control group, mice did not have applied dressings, while in commercial gel and the fiber-based hydrogel with/without drug group, the mice were dressed with the corresponding hydrogels respectively. The healing results at various times are shown in FIG. 8a. Photographs of mice skin wound tissues on days 0, 4, 8, and 12 in the control group, the commercial gel group (Hydrosorb® Gel), the fiber-based gel without drug and the fiber-based gel with drug group show that the wound site of our fiber-based hydrogel is substantially reduced. The inner diameter of the rubber ring is 1 cm. Measured wound healing rates of different groups for 4, 8, and 12 days are shown in FIG. 8b, indicating that the fastest wound healing rates occur with the fiber-based hydrogel. In conclusion, as evidenced by the macro-observations, the fiber-based hydrogel exhibited significant potential to expedite tissue regeneration.

In one embodiment, the fiber-based hydrogel contains from 1% to 7% by dry weight biocompatible microfibers having a high aspect ratio suspended in from 93% to 99% by weight water. In another embodiment, the fiber-based hydrogel contains from 2% to 6% by dry weight biocompatible microfibers having a high aspect ratio length suspended in from 94% to 98% by weight water. In yet another embodiment, the fiber-based hydrogel contains from 3% to 5% by dry weight biocompatible microfibers having a high aspect ratio length suspended in from 95% to 97% by weight water.

The fiber-based hydrogel has a porosity that facilitates convenient, beneficial, and/or safe use with biological systems. In one embodiment, the fiber-based hydrogel has a porosity from 5% to 30%. In another embodiment, the fiber-based hydrogel has a porosity from 10% to 25%. For example, therapeutics and/or biological reagents (antibiotics, epidermal growth factors, other active ingredients) can be included within the fiber-based hydrogel.

The fiber-based hydrogels described herein can be used, for example, in liquid bandage products, bandage products, and wound healing treatments.

Following are examples that illustrate procedures for practicing the invention. These examples should not be construed as limiting.

### Example 1A Fabrication of Fiber-Based Hydrogel by Using Polyethylene Glycol Diacrylate (PEGDA)

Microfibers are made of polyethylene glycol diacrylate (PEGDA) that can be polymerized by ultraviolet (UV) light in a flow-focusing microfluidic channel, as shown in FIG. 1a. After UV exposure, the microfibers are successfully synthesized, as shown in FIG. 1b. The diameter of the fiber is 100 μm and the ratio of fiber length to fiber diameter, L/D=600. By turning the UV curing time and flow rates, microfibers with different aspect ratios (L/D=80-2000) can be produced.

After collecting a certain volume of microfiber, as shown in FIG. 2a, the microfibers are washed with deionized water. The injectable hydrogel is fabricated by extruding the microfibers suspension from a syringe with/without a needle, as shown in FIGS. 2b and 2c.

### Example 1B Fabrication of Fiber-Based Hydrogel by Using N-Isopropylacrylamide (NIPAM)

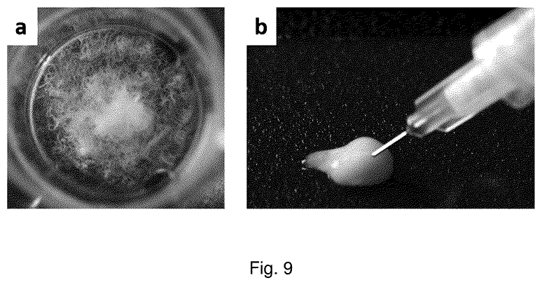

Microfibers are made of N-Isopropylacrylamide (NIPAM) that can be polymerized by ultraviolet (UV) light in a flow-focusing microfluidic channel, the same as Example 1A. After UV exposure, the microfibers having a high respect ratio (L/D=400) are successfully synthesized, as shown in FIG. 9a. The PNIPAM fiber-based hydrogel is produced immediately after extrusion as shown in FIG. 9b.

### Example 2 Fabrication of Fiber-Based Hydrogel with Drug

The polymer solution is mixed with drugs, 1 mg/mL tetracycline (TC) and 0.5 ng/ml epidermal growth factor (EGF). The microfibers with drugs are fabricated by UV illumination in a flow-focusing microfluidic channel. The drug hydrogels are made by extruding the drug-containing microfibers suspension from a syringe.

Unless otherwise indicated in the examples and elsewhere in the specification and claims, all parts and percentages are by weight, all temperatures are in degrees Centigrade, and pressure is at or near atmospheric pressure.

With respect to any figure or numerical range for a given characteristic, a figure or a parameter from one range may be combined with another figure or a parameter from a different range for the same characteristic to generate a numerical range.

Other than in the operating examples, or where otherwise indicated, all numbers, values and/or expressions referring to quantities of ingredients, reaction conditions, etc., used in the specification and claims are to be understood as modified in all instances by the term “about.”

While the invention is explained in relation to certain embodiments, it is to be understood that various modifications thereof will become apparent to those skilled in the art upon reading the specification. Therefore, it is to be understood that the invention disclosed herein is intended to cover such modifications as fall within the scope of the appended claims.

1. A fiber-based hydrogel, comprising:
an aqueous hydrogel comprising from 1% to 7% by dry weight biocompatible microfibers having a high aspect ratio length to diameter of at least 100 in from 93% to 99% by weight water, the biocompatible microfibers mechanically interlocked.

2. The fiber-based hydrogel according to claim 1, wherein the biocompatible microfibers have a high aspect ratio length to diameter of at least 400.

3. The fiber-based hydrogel according to claim 1, wherein the biocompatible microfibers have a high aspect ratio length to diameter of at least 2000.

4. The fiber-based hydrogel according to claim 1, wherein the biocompatible microfibers comprise a hydrophilic polymer.

5. The fiber-based hydrogel according to claim 1, comprising from 2% to 6% by dry weight biocompatible microfibers and from 94% to 98% by weight water.

6. The fiber-based hydrogel according to claim 1, with the proviso that the different biocompatible microfibers are not chemically cross-linked amongst each other.

7. The fiber-based hydrogel according to claim 1, having a porosity from 5% to 30%.

8. The fiber-based hydrogel according to claim 1, with the proviso that the fiber-based hydrogel does not comprise a surfactant.

9. The fiber-based hydrogel according to claim 1, with the proviso that the fiber-based hydrogel does not comprise an organic solvent.

10. A method of making a fiber-based hydrogel, comprising:
contacting an aqueous polymer phase comprising a photoinitiator with an aqueous solution phase under ultraviolet light exposure and suitable shear forces to generate biocompatible microfibers having a high aspect ratio length to diameter of at least 100; and
mechanically interlocking from 1% to 7% by dry weight of the biocompatible microfibers in from 93% to 99% by weight water to provide the fiber-based hydrogel.

11. The method according to claim 10, wherein the aqueous polymer phase is contacted with the aqueous solution phase in a syringe.

12. The method according to claim 10, wherein the aqueous polymer phase is contacted with the aqueous solution phase in a flow-focusing microfluidic device.

13. The method according to claim 10, with the proviso that the aqueous polymer phase is contacted with the aqueous solution phase in the absence of a surfactant.

14. The method according to claim 10, with the proviso that the aqueous polymer phase is contacted with the aqueous solution phase in the absence of an organic solvent.

15. A method of treating a wound, comprising:
applying to a wound a fiber-based hydrogel, comprising an aqueous hydrogel comprising from 1% to 7% by dry weight biocompatible microfibers having a high aspect ratio length to diameter of at least 100 in from 93% to 99% by weight water, the biocompatible microfibers mechanically interlocked.

16. A method of making a fiber-based hydrogel with tunable mechanical properties and/or drug release profile, comprising:
contacting an aqueous polymer phase comprising a photoinitiator with an aqueous solution phase under ultraviolet light exposure and suitable shear forces to generate biocompatible microfibers having a high aspect ratio length to diameter of at least 100; and
mechanically interlocking from 1% to 7% by dry weight of the biocompatible microfibers in from 93% to 99% by weight water to provide the fiber-based hydrogel.

17. The method according to claim 16, wherein the fiber aspect ratio is adjusted to tune a considerable range of mechanical properties and/drug release profile.

18. A method of making a medicament with controlled drug release rate and/or drug release profile, comprising:
using the fiber-based hydrogel of claim 1 in the medicament.

19. A method of making a medicament with controlled drug release rate and/or drug release profile, comprising:
using the fiber-based hydrogel of claim 1 in the medicament.
